SEQUENCE LISTING (16 sequences)

SEQ ID NO: 1 - protein, 8 aa, Artificial Sequence
  Synthetic - antibody 2F1; CDR1 VH
GFSLTDYG

SEQ ID NO: 2 - protein, 7 aa, Artificial Sequence
  Synthetic - antibody 2F1; CDR2 VH
IWGDGST

SEQ ID NO: 3 - protein, 11 aa, Artificial Sequence
  Synthetic - antibody 2F1; CDR3 VH
AGALYDGYYGY

SEQ ID NO: 4 - protein, 6 aa, Artificial Sequence
  Synthetic - antibody 2F1; CDR1 VL
QDVSTA

SEQ ID NO: 5 - skipped

SEQ ID NO: 6 - protein, 9 aa, Artificial Sequence
  Synthetic - antibody 2F1; CDR3 VL
QQHYSTPLT

SEQ ID NO: 7 - protein, 8 aa, Artificial Sequence
  Synthetic - antibody 3A10; CDR1 VH
GLTFSDAW

SEQ ID NO: 8 - protein, 10 aa, Artificial Sequence
  Synthetic - antibody 3A10; CDR2 VH
IRSKAHNHAT

SEQ ID NO: 9 - protein, 9 aa, Artificial Sequence
  Synthetic - antibody 3A10; CDR3 VH
TRYGNPFVY

SEQ ID NO: 10 - protein, 13 aa, Artificial Sequence
  Synthetic - antibody 3A10; CDR1 VL
SPSLLYSSNQKNY

SEQ ID NO: 11 - skipped

SEQ ID NO: 12 - protein, 9 aa, Artificial Sequence
  Synthetic - antibody 3A10; CDR3 VL
QQYYSYPWT

SEQ ID NO: 13 - protein, 117 aa, Artificial Sequence
  Synthetic - antibody 2F1; VH
EVQLEESGPGLVAPSQSLSITCTVSGFSLTDYGVTWIRQPPGKGLEWLGVIWGDGSTYYNSALKSRLSISKDDSKSQVFLKMNSLQTDDTAMYYCAGALYDGYYGYWGQGTTLTVSS

SEQ ID NO: 14 - protein, 107 aa, Artificial Sequence
  Synthetic - antibody 2F1; VL
DIVLTQTHKFMSTSVGDRVSITCKASQDVSTAVAWYQQKPGQSPKLLIYSASYRYSGVPDRFTGSGSGTDFTFTISSVQAEDLAVYYCQQHYSTPLTFGAGTKLELK

SEQ ID NO: 15 - protein, 118 aa, Artificial Sequence
  Synthetic - antibody 3A10; VH
EVQLQQSGGGLVQPGGSMKLSCAASGLTFSDAWMDWVRQSPEKGLEWVAEIRSKAHNHATYYAESVKGRFTISRDDSKSSVYLQMNSLRAEDTGIYYCTRYGNPFVYWGQGTLVTVSA

SEQ ID NO: 16 - protein, 113 aa, Artificial Sequence
  Synthetic - antibody 3A10; VL
DIVMTQSPSSLAVSVGEKVTMSCKSSPSLLYSSNQKNYLAWYQQKPGQSPKLLIYWASTRESGVPDRFTGSGSGTDFTLTISSVKAEDLAVYYCQQYYSYPWTFGGGTKLEIK

(19) United States
(12) Patent Application Publication
(10) Pub. No.: US 2024/0026034 A1
(43) Pub. Date: Jan. 25, 2024

(54) ANTIBODIES SPECIFIC FOR QSOX1 AND METHODS OF USING THE SAME

(71) Applicants: ARIZONA BOARD OF REGENTS ON BEHALF OF ARIZONA STATE UNIVERSITY, Scottsdale, AZ (US); MAYO FOUNDATION FOR MEDICAL EDUCATION AND RESEARCH, Rochester, MN (US)

(72) Inventors: Douglas LAKE, Scottsdale, AZ (US); Thai HO, Scottsdale, AZ (US); Calvin KOELBEL, Mesa, AZ (US)

(21) Appl. No.: 18/257,444

(22) PCT Filed: Dec. 15, 2021
(86) PCT No.: PCT/US2021/063442

§ 371 Date: Jun. 14, 2023

Related U.S. Application Data
(60) Provisional application No. 63/125,961, filed on Dec. 15, 2020.

(51) Int. Cl. C07K 16/40; A61P 35/00; G01N 33/573

(52) U.S. Cl. CPC C07K 16/40; A61P 35/00; G01N 33/573; C07K 2317/565; C07K 2317/92; C07K 2317/567; C07K 2317/52; C07K 2317/24

(57) ABSTRACT

Provided are antibodies specific for human QSOX1 and methods of using these antibodies or antigen binding regions thereof in assays to detect QSOX1, to inhibit QSOX1, for use in other assay systems, and for use in treating cancer.

### CROSS-REFERENCE TO RELATED APPLICATION

This application claims the benefit of U.S. Provisional Application Ser. No. 63/125,961, filed Dec. 15, 2020, which is incorporated by reference herein as if set forth in its entirety.

### STATEMENT REGARDING FEDERALLY SPONSORED RESEARCH

This invention was made with government support under R01 CA201226 awarded by the National Institutes of Health. The government has certain rights in the invention.

### SEQUENCE LISTING

The instant application contains a Sequence Listing which has been electronically submitted in ASCII format and is hereby incorporated by reference in its entirety. Said ASCII copy, created on Dec. 14, 2021, is named 112624_01314_ST25.txt and is 6,880 bytes in size.

### BACKGROUND OF THE INVENTION

Despite systemic treatment, metastases are the primary cause of mortality in cancer, and the molecular mechanisms in the extracellular matrix (ECM) that drive metastasis are poorly understood. ECM components are upregulated in cancer metastases when compared to matched primary tumors. Current cancer therapies already target angiogenesis, tyrosine kinases, DNA repair, and immune checkpoints, but to date a pharmaceutical targeting a cancer cell-ECM interaction has not been approved by the FDA. QSOX1, a sulfhydryl oxidase, is upregulated in cancer and is capable of facile generation/shuffling of disulfides in proteins. There is a need to develop tools that interrogate QSOX1 levels for diagnostic purposes. Further, therapeutic targeting of QSOX1 offers a unique opportunity to simultaneously target multiple disulfide-bonded ECM proteins simultaneously with a single agent instead of focusing on individual ECM components. There is a need to develop agents that target QSOX1 for medical benefit. Because metastases are the primary cause of renal cell carcinoma (RCC) mortality, there is a particular need to develop therapeutics that target the extracellular matrix (ECM) or a renal carcinoma cell-ECM interaction for treating RCC.

### SUMMARY OF THE INVENTION

Provided herein are anti-QSOX1 antibodies, each comprising three heavy chain CDRs: VH CDR1, VH CDR2, and VH CDR3, and three light chain CDRs: VL CDR1, VL CDR2, and VL CDR3, wherein the CDRs comprise a sequence selected from any one of SEQ ID NOs: 1-12. In some embodiments, the anti-QSOX1 antibody comprises a heavy chain variable region and a light chain variable region, wherein the heavy chain variable region (“VH”) comprises three complementarity determining regions, VH CDR1, VH CDR2, and VH CDR3 and the light chain variable region (“VL”) comprises three complementarity determining regions, VL CDR1, VL CDR2, and VL CDR3; and wherein the VH CDR1 comprises a sequence selected from the group consisting of SEQ ID NO: 1, SEQ ID NO: 7, and a sequence having at least 87% identity to SEQ ID NO: 1 or SEQ ID NO: 7; the VH CDR2 comprises a sequence selected from the group consisting of SEQ ID NO: 2, SEQ ID NO: 8, and a sequence having at least 85% identity to SEQ ID NO: 2 or SEQ ID NO: 8; the VH CDR3 comprises a sequence selected from the group consisting of SEQ ID NO: 3, SEQ ID NO: 9, and a sequence having at least 88% identity to SEQ ID NO: 3 or SEQ ID NO: 9; the VL CDR1 comprises a sequence selected from the group consisting of SEQ ID NO: 4, SEQ ID NO: 10, and a sequence having at least 83% identity to SEQ ID NO: 4 or SEQ ID NO: 10; the VL CDR2 comprises a sequence selected from the group consisting of SEQ ID NO: 5 and SEQ ID NO: 11; and the VL CDR3 comprises a sequence selected from the group consisting of SEQ ID NO: 6, SEQ ID NO: 12, and a sequence having at least 88% identity to SEQ ID NO: 6 or SEQ ID NO: 12. In some embodiments, the antibody binds to human QSOX1, or a fragment thereof, with an affinity characterized by a KD less than 10⁻⁶ M. In some embodiments, the antibody comprises a VH comprising SEQ ID NO: 13, SEQ ID NO: 15, or a sequence having at least 90% identity to the aforementioned. In some embodiments, the antibody comprises a VL comprising SEQ ID NO: 14, SEQ ID NO: 16, or a sequence having at least 90% identity to the aforementioned.

Further provided herein is a composition comprising an antibody described herein and an additional agent, such as, e.g., a pharmaceutically acceptable carrier or excipient.

In another aspect, provided herein are methods of using an antibody described herein. These include a method for treating cancer or initiating, enhancing, or prolonging an anti-cancer response in an individual, wherein the method comprises administering a therapeutically effective amount of an antibody provided herein to a subject in need thereof.

These methods also include methods of determining the level of QSOX1 in a sample using an antibody described herein. These include a method comprising obtaining a sample from an individual and determining a level of QSOX1 in the sample using an antibody described herein. The sample may be a blood or plasma sample. The determining step may comprise an antigen capture assay comprising the antibody, such as the antibody linked to a detectable label. In some embodiments, the antibody is attached to a solid support and incubating with the sample. In some embodiments, the method of determining the level of QSOX1 in a sample is a method of detecting cancer in an individual.

In a still further aspect, the method of detecting cancer in an individual comprises obtaining a sample from the individual and determining the level of QSOX1 in the sample using at least one of the antibodies provided herein, where an increase in QSOX1 in the sample as compared to a control is indicative of cancer in the subject. In some embodiments, a level of QSOX1 of greater than 5,000 ng/mL is indicative of the presence of a cancer in the individual.

Other aspects, objects and features of the invention will be apparent from the following description.

### BRIEF DESCRIPTION OF THE DRAWINGS

FIG. 1 shows data from the development and screening of anti-QSOX1 antibodies. FIG. 1 shows results from screening multiple clones for the ability to bind QSOX1 and inhibit QSOX1 enzymatic activity. FIG. 1-A shows results of screening antibody clones for QSOX1-binding in a fluorescence-based binding assay. 10 nM QSOX1 and 30 μL hybridoma supernatant was pre-incubated for 10 minutes followed by addition of the mixture of components of the assay. Decreased fluorescence is associated with inhibition of QSOX1 enzymatic activity. 300 μM DTT was used as a substrate. Positive control was 35 μM H2O2. Negative control was 100 nM QSOX1 with no substrate. FIG. 1-B is a photograph of a Western blot showing QSOX1 binding to different antibodies. The arrows indicate binding of monoclonal antibodies (mAb's) to recombinant QSOX1. FIG. 1-C is a graph showing concentration-dependent inhibition of QSOX1 activity by various antibodies purified using Protein A. FIG. 1-C shows that 105.F11 is a less inhibitory antibody than 2F1.E2 and 1B8.F7.

FIG. 2: QSOX1 antigen capture assay using anti-QSOX1 antibodies detecting QSOX1 in plasma samples. FIG. 2 shows a graph showing the ability to detect increased QSOX1 in plasma from individuals with cancer as elevated compared to normal controls and a diagram of a QSOX1 antigen capture assay described herein. The coating mAb was 3A10 and the detection mAb was 2F1.F4-biotin. Note: coating mAb is 3A10 and detection mAb is 2F1.F4-biotin.

FIG. 3: 2F1.14 binds at the first CxxC motif (70-73) in QSOX1. FIG. 3 shows a series of binding assays demonstrating that the binding of QSOX1 by the antibody 2F1 requires the C-X-X-C motif at 70-73 on the left side as compared to loading controls on the right side. The 3A10 antibody binds to a distinct site and is not affected by mutations at C70 or C73 of QSOX1.

FIG. 4: 3D Invasion Assay Workflow. FIG. 4 shows a diagram showing the 3D invasion assay used in the examples.

FIG. 5: 2F1.14 suppresses 3D invasion, other antibodies do not. FIG. 5 shows the results of the 3D invasion assay for the antibody 2F1.14, 3A10.6 and a control mouse IgG. Antibody 2F1 suppressed 3D invasion whereas other antibodies did not. The numbers above the error bars indicate the number of spheroid replicates included in the analysis.

### DETAILED DESCRIPTION OF THE INVENTION

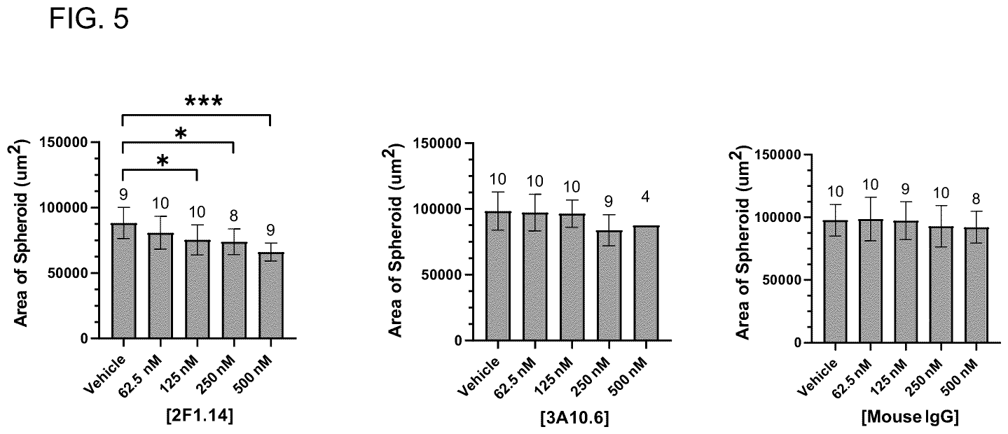

QSOX1 is over-expressed in multiple different tumor types, thus detecting and measuring QSOX1 levels in plasma could provide a diagnostic tool for monitoring tumor burden. Additionally, since the enzymatic activity of QSOX1 plays a role in tumor invasion and metastasis, mAbs inhibiting QSOX1 in the ECM may suppress metastasis. The inventors generated multiple murine monoclonal antibodies specific for QSOX1. The present invention is based on part on the discovery of antibodies that bind and inhibit QSOX1. Using recombinant QSOX1 protein generated in a eukaryotic expression system, mAbs were generated that inhibit QSOX1 enzymatic activity in an in vitro fluorescence assay. Furthermore, a pair of these mAbs (2F1 and 3A10) detected elevated levels of QSOX1 in human plasma in cancer patients and lower levels in control plasma from normal donors. The data herein demonstrates detecting QSOX1 as a companion diagnostic and the potential of inhibitory QSOX1 mAbs for therapeutic applications. The potential use in therapeutic applications was demonstrated based on the assay shown in FIG. 5 which demonstrates that at least the 2F1 antibody may be able to suppress 3D invasion and may be useful in inhibiting cancer or inhibiting metastases of the cancer.

The antigen capture assay to detect QSOX1 would be helpful for diagnosis in cases for which imaging may show a cyst or some fluid secondary to a potential malignancy. Additionally, if a patient is undergoing treatment of a QSOX1-positive tumor which has been shown to secrete QSOX1 into circulation, detecting QSOX1 in circulating blood plasma would be useful to know if a particular treatment is reducing tumor burden in the patient. Both antibodies provided herein may be used for such assays.

In addition, the antibodies described herein may be useful in other assays as controls. For example, 3A10 was used as control mAb that does not specifically bind to any neutralizing antibody to SARS-CoV-2 in a Lateral Flow Assay that measures levels of neutralizing antibodies to SARS-CoV-2.

While the terms used in the description of the invention are believed to be well understood by one of ordinary skill in the pharmaceutical arts, definitions, where provided herein, are set forth to facilitate description of the invention, and to provide illustrative examples for use of the terms.

As used herein, the terms “a”, “an”, and “the” mean “one or more”, unless the singular is expressly specified (e.g., singular is expressly specified, for example, in the phrase “a single formulation”).

The terms “specifically binds”, or “binding specificity” are used alternatively and in relation to an antibody, refers to the ability of the antibody to form one or more noncovalent bonds with an epitope or antigen (e.g., an antigen exemplified herein includes human QSOX1) via the antibody variable domains. Specificity can be characterized by an antibody-antigen affinity, e.g. as characterized by a dissociation constant (KD) of <100 nM, 10 nM, <1 nM, <0.1 nM, <0.01 nM, or <0.001 nanomolar (nM) (e.g. 10⁻⁸ M or less, e.g. from 10⁻⁸ M to 10⁻¹³ M, e.g., from 10⁻⁹ M to 10⁻¹³ M).

The term “identity”, as recognized by those skilled in the art, represents a comparison between two or more amino acid sequences performed using published methods and software known in the art. For example, the compared amino acid sequences are optimally aligned, and the number of amino acid differences are counted and converted to a percentage. For example, if a first amino acid sequence of 50 amino acids is optimally aligned with a second amino acid sequence of 50 amino acids, and 5 out of 50 amino acids differ from the second amino acid sequence, then the first amino acid sequence is said to have 10% identity with the second amino acid sequence.

The term “antibody” refers to a full-length antibody, derivatives or fragments of full length antibodies that comprise less than the full-length sequence of the antibody but retain at least the binding specificity of the full-length antibody (e.g., variable portions of the light chain and heavy chain), chimeric antibodies, humanized antibodies, synthetic antibodies, recombinantly produced antibodies, as known to those skilled in the art, and produced using methods known in the art. An antibody of a particular IgG class may be referred to by its subclass (e.g., IgG1, IgG2, IgG3, and IgG4). Amino acid sequences are known to those skilled in the art for the Fc portion of antibodies of the respective IgG subclass. Examples of antibody fragments include, but are not limited to, Fab, Fab′, F(ab′)₂, scFv, Fv, dimeric scFv, Fd, and Fd. Fragments may be synthesized or generated by enzymatic cleavage using methods known in the art. Antibodies can also be produced in either prokaryotic or eukaryotic in vitro translation systems using methods known in the art.

As used herein, the term “heavy chain” (VH) or “light chain” (VL) respectively refer to an antibody variable heavy (VH) and variable light (VL) domain consisting of a “framework” region interrupted by the three CDRs. The complementarity-determining regions (CDRs) of an antibody are subregions of the variable chains in antibodies involved in binding specific antigens. An antibody may be referred to by the antibody's CDRs, which is typically six CDRs total. Thus, an antibody may be referred to herein by the antibody's CDRs of the heavy chain (V_{H} CDR 1, V_{H} CDR 2, and V_{H} CDR 3) and the light chain (V_{L} CDR 1, V_{L} CDR 2, and V_{L} CDR 3).

Antibodies herein specifically include “chimeric” antibodies (immunoglobulins), as well as fragments of such antibodies, as long as they exhibit the desired biological activity (U.S. Pat. No. 4,816,567; Morrison et al., Proc. Natl. Acad. Sci. USA 81: 6851-6855 (1984); Oi et al., Biotechnologies 4(3): 214-221 (1986); and Liu et al., Proc. Natl. Acad. Sci. USA 84: 3439-43 (1987)).

“Humanized” or “CDR grafted” forms of non-human (e.g., murine) antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. The term “hypervariable region” when used herein refers to the amino acid residues of an antibody which are associated with its binding to antigen. The hypervariable regions encompass the amino acid residues of the “complementarity determining regions” or “CDRs”. In some instances, framework region (FW) residues of the human immunoglobulin are also replaced by corresponding non-human residues (so called “back mutations”). Furthermore, humanized antibodies may be modified to comprise residues which are not found in the recipient antibody or in the donor antibody, in order to further improve antibody properties, such as affinity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321: 522-525 (1986); and Reichmann et al., Nature 332: 323-329 (1988).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger el al., Proc. Natl. Acad. Sci. USA 90: 6444-6448 (1993).

The expression “linear antibodies” when used throughout this application refers to the antibodies described in Zapata, et al. Protein Eng. 8(10): 1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Variable regions and CDR sequences in an antibody can be identified according to general rules that have been developed in the art, such as, as set out above, for example using the Kabat nomenclature system, or by aligning the sequences against a database of known variable regions. CDRs can be defined using different systems such as, e.g., Kabat, Chothia, Kabat/Chothia, McCallum/Contact, IMGT, Gelfand, Honneger, Martin, North, and AbM.

The term “pharmaceutically acceptable carrier” is used herein to mean any compound or composition or carrier medium useful in any one or more of administration, delivery, storage, stability of a composition or combination described herein. These carriers are known in the art to include, but are not limited to, a diluent, water, saline, suitable vehicle (e.g., liposome, microparticle, nanoparticle, emulsion, capsule), buffer, medical parenteral vehicle, excipient, aqueous solution, suspension, solvent, emulsions, detergent, chelating agent, solubilizing agent, salt, colorant, polymer, hydrogel, surfactant, emulsifier, adjuvant, filler, preservative, stabilizer, oil, binder, disintegrant, absorbant, flavor agent, and the like as broadly known in the pharmaceutical art.

### Anti-QSOX1 Antibodies

Provided herein are anti-QSOX1 antibodies, each comprising three heavy chain CDRs: VH CDR1, VH CDR2, and VH CDR3, and three light chain CDRs: VL CDR1, VL CDR2, and VL CDR3, wherein the CDRs comprise a sequence selected from any one of SEQ ID NOs: 1-12. An Anti-QSOX1 antibody is an antibody or fragment thereof capable of binding to QSOX1.

Antibodies capable of binding QSOX1 are provided. An antibody that specifically binds to QSOX1 may comprise a heavy chain variable region and a light chain variable region, wherein the heavy chain variable region (“VH”) comprises three complementarity determining regions, VH CDR1, VH CDR2, and VH CDR3 and the light chain variable region (“VL”) comprises three complementarity determining regions, VL CDR1, VL CDR2, and VL CDR3, and wherein the VH CDR1 comprises a sequence selected from the group consisting of SEQ ID NO: 1, SEQ ID NO: 7, and a sequence having at least 80%, 83%, 85%, 87%, 88%, 90%, 92%, 95%, 96%, 97%, 98% identity to SEQ ID NO: 1 or SEQ ID NO: 7; the VH CDR2 comprises a sequence selected from the group consisting of SEQ ID NO: 2, SEQ ID NO: 8, and a sequence having at least 80%, 83%, 85%, 87%, 88%, 90%, 92%, 95%, 96%, 97%, 98% identity to SEQ ID NO: 2 or SEQ ID NO: 8; the VH CDR3 comprises a sequence selected from the group consisting of SEQ ID NO: 3, SEQ ID NO: 9, and a sequence having at least 80%, 83%, 85%, 87%, 88%, 90%, 92%, 95%, 96%, 97%, 98% identity to SEQ ID NO: 3 or SEQ ID NO: 9; the VL CDR1 comprises a sequence selected from the group consisting of SEQ ID NO: 4, SEQ ID NO: 10, and a sequence having at least 80%, 83%, 85%, 87%, 88%, 90%, 92%, 95%, 96%, 97%, 98% identity to SEQ ID NO: 4 or SEQ ID NO: 10; the VL CDR2 comprises a sequence selected from the group consisting of SEQ ID NO: 5, SEQ ID NO: 11, and a sequence having at least 66%, 80%, 83%, 85%, 87%, 88%, 90%, 92%, 95%, 96%, 97%, 98% identity to SEQ ID NO: 5 or SEQ ID NO: 11; and the VL CDR3 comprising a sequence selected from the group consisting of SEQ ID NO: 6, SEQ ID NO: 12, and a sequence having at least 80%, 83%, 85%, 87%, 88%, 90%, 92%, 95%, 96%, 97%, 98% identity to SEQ ID NO: 6 or SEQ ID NO: 12.

In some embodiments, the antibody comprises the VH CDR1 comprising SEQ ID NO: 1 or a sequence having at least 66%, 80%, 83%, 85%, 87%, 88%, 90%, 92%, 95%, 96%, 97%, 98% identity thereto, the VH CDR2 comprising SEQ ID NO: 2 or a sequence having at least 66%, 80%, 83%, 85%, 87%, 88%, 90%, 92%, 95%, 96%, 97%, 98% identity thereto, the VH CDR3 comprising SEQ ID NO: 3 or a sequence having at least 66%, 80%, 83%, 85%, 87%, 88%, 90%, 92%, 95%, 96%, 97%, 98% identity thereto, the VL CDR1 comprising SEQ ID NO: 4 or a sequence having at least 66%, 80%, 83%, 85%, 87%, 88%, 90%, 92%, 95%, 96%, 97%, 98% identity thereto, the VL CDR2 comprising SEQ ID NO: 5 or a sequence having at least 66%, 80%, 83%, 85%, 87%, 88%, 90%, 92%, 95%, 96%, 97%, 98% identity thereto, the VL CDR 3 comprising SEQ ID NO: 6 or a sequence having at least 66%, 80%, 83%, 85%, 87%, 88%, 90%, 92%, 95%, 96%, 97%, 98% identity thereto.

In some embodiments, the antibody comprises the VH CDR1 comprising SEQ ID NO: 7 or a sequence having at least 66%, 80%, 83%, 85%, 87%, 88%, 90%, 92%, 95%, 96%, 97%, 98% identity thereto, the VH CDR2 comprising SEQ ID NO: 8 or a sequence having at least 66%, 80%, 83%, 85%, 87%, 88%, 90%, 92%, 95%, 96%, 97%, 98% identity thereto, the VH CDR3 comprising SEQ ID NO: 9 or a sequence having at least 66%, 80%, 83%, 85%, 87%, 88%, 90%, 92%, 95%, 96%, 97%, 98% identity thereto, the VL CDR1 comprising SEQ ID NO: 10 or a sequence having at least 66%, 80%, 83%, 85%, 87%, 88%, 90%, 92%, 95%, 96%, 97%, 98% identity thereto, the VL CDR2 comprising SEQ ID NO: 11 or a sequence having at least 66%, 80%, 83%, 85%, 87%, 88%, 90%, 92%, 95%, 96%, 97%, 98% identity thereto, the VL CDR 3 comprising SEQ ID NO: 12 or a sequence having at least 66%, 80%, 83%, 85%, 87%, 88%, 90%, 92%, 95%, 96%, 97%, 98% identity thereto.

Any of the CDRs or CDR sets described herein may be used to generate humanized antibodies by combination with FW regions and constant regions of human antibodies to generate QSOX1 humanized antibodies. The CDRs are placed within FW regions such that the heavy chain variable region (“VH”) which comprises three complementarity determining regions, VH CDR1, VH CDR2, and VH CDR3, and four framework regions, VH FW1, VH FW2, VH FW3, and VH FW4, are present in the order VH FW1-VH CDR1-VH FW2-VH CDR2-VH FW3-VH CDR3-VH FW4 and the light chain variable region (“VL”) which also comprises three complementarity determining regions, VL CDR1, VL CDR2, and VL CDR3, and four framework regions, VL FW1, VL FW2, VL FW3, and VL FW4, are present in the order VL FW1-VL CDR1-VL FW2-VL CDR2-VL FW3-VL CDR3-VL FW4. Those skilled in the art are capable of generating humanized antibodies based on the QSOX1 specific CDRs or heavy and light chain variable regions provided herein.

In some embodiments, the antibody comprises the VH comprising or consisting of the sequence selected from the group consisting of SEQ ID NO: 13, SEQ ID NO: 15, and a sequence having at least 80%, 85%, 90%, 92%, 95%, 96%, 97%, 98% identity to SEQ ID NO: 13 or SEQ ID NO: 15 and the VL comprising or consisting of the sequence selected from the group consisting of SEQ ID NO: 14, SEQ ID NO: 16, and a sequence having at least 80%, 85%, 90%, 92%, 95%, 96%, 97%, 98% identity to SEQ ID NO: 14 or SEQ ID NO: 16. Suitably, some embodiments of the antibody comprise SEQ ID NOs: 13 and 14 or SEQ ID NOs: 15 and 16. In other embodiments, the antibody comprises SEQ ID NOs: 13 and 16 or SEQ ID NOs: 14 and 15.

The sequences provided are only for the variable regions of the antibody. Those skilled in the art will appreciate that these regions determine the specificity of the antibody but that the effector function of the antibody is generally dependent on the constant regions (and the specific isotype) of the antibody. Those skilled in the art can engineer antibodies for specific purposes based on the variable regions provided herein. In some embodiments, the antibody comprises an Fc portion of a human or humanized antibody. In some embodiments, the antibody comprises an Fc region which has been engineered for a suitable effector function(s).

Provided here are anti-QSOX1 antibodies comprising a detectable label, such as a label described herein and/or known to the skilled worker. Non-limiting examples of detectable labels include fluorophores (e.g. GFPs and fluoresceins), horseradish peroxidases, alkaline phosphatases, luminescent agents, luciferase, radioisotopes, biochemical tags (e.g. biotin or avidin), and colorimetric reagents.

Provided here is an anti-QSOX1 antibody attached to a solid support. An antibody described herein may be immobilized to a solid support known in the art by technique known to the skilled worker. Non-limiting examples of a solid support include substrates, polymers, beads, microarrays, microtiter plates, slides, tissue culture plates, and tissue culture wells.

Provided here are anti-QSOX1 antibodies that inhibit an enzymatic activity of QSOX1, including, e.g., human QSOX1, such as in an assay described herein and/or known to the skilled worker.

Provided herein are antibodies that reduce or inhibit 3D invasion of a cancer cell in vitro, such as, e.g., in an assay described herein and/or known to the skilled worker.

### Compositions Comprising an Anti-QSOX1 Antibody

Provided herein are compositions comprising an antibody described herein and an additional agent. These compositions include pharmaceutical and/or diagnostic compositions. In some embodiments the additional agent is a pharmaceutically acceptable carrier or excipient. In some embodiments, a composition of the invention comprises an antibody described herein and a pharmaceutically acceptable carrier or excipient described in Remington's Pharmaceutical Sciences 16th edition, Osol, A. Ed. (1980) and/or otherwise known in the art. Preferably, the pharmaceutically acceptable carrier is a liquid suitable for intravenous administration and/or injection of an anti-QSOX1 antibody.

### Methods of Using an Anti-QSOX1 Antibody

An antibody capable of binding QSOX1 described herein may be used as the antibody in any of the methods described herein. Such methods include methods of treating a disease or disorder in a subject and methods of determining the level of QSOX1 in a sample. In some embodiments, the disease or disorder is a cancer. In some embodiments, the sample is from a subject, such as, e.g., a blood and/or plasma sample.

Provided here are methods of using an antibody described herein or a composition thereof. These include a method for treating cancer or initiating, enhancing, or prolonging an anti-cancer response in an individual, wherein the method comprises administering a therapeutically effective amount of an antibody provided herein, or a composition thereof, to a subject in need thereof. In some embodiments of the methods described herein, the antibody comprises a detectable label.

Provided here are methods of determining the level of QSOX1 in a sample using an antibody described herein. These include a method comprising obtaining a sample from an individual and determining a level of QSOX1 in the sample using an antibody described herein. The sample may be a blood or plasma sample. The determining step may comprise an antigen capture assay comprising the antibody, such as the antibody linked to a detectable label. In some embodiments, the antibody is attached to a solid support and incubating with the sample.

In some embodiments, the level of QSOX1 in a sample will be correlated with a disease or condition in the individual from which the sample was derived. Thus, a determined level of QSOX1 from a sample can provide information, e.g., diagnostic, prognostic and/or therapeutic response information, about a subject, including, such as, whether the subject would benefit from a particular therapeutic modality, survival prospects, and/or whether the subject would benefit from additional screening for a particular cancer or additional diagnostic method.

In some embodiments, the method of determining the level of QSOX1 in a sample can be used as a method of detecting or predicting the presence of a cancer in an individual. In some embodiments, the method of diagnosis cancer in an individual comprises obtaining a sample from the individual and determining the level of QSOX1 in the sample using at least one of the antibodies provided herein, where an increase in QSOX1 in the sample as compared to a control is indicative of cancer in the subject. In some embodiments, a level of QSOX1 of greater than 500 ng/mL, 750 ng/mL, 1,000 ng/mL, 1,250 ng/mL, 1,500 ng/mL, 3,000 ng/mL, 4,000 ng/mL, 5,000 ng/mL, 7,500 ng/mL, 10,000 ng/mL, and/or 15,000 ng/mL is indicative of the presence of a cancer in the individual. In some embodiments, the sample is a blood sample and/or a plasma sample.

Provided here are methods of diagnosing and treating a patient, the method comprising (1) determining the level of QSOX1 in a sample from the patient using an antibody described herein, or a composition thereof, and subsequently (2) treating the patient with antibody described herein.

The terms “treat”, “treating”, or “treatment” as used herein, embrace one or more of preventative (prophylactically) or therapeutically (palliative). As used herein, the terms “treat”, “treating”, or “treatment” include but are not limited to initiating, enhancing, or prolonging an anti-cancer response. In treatment of cancer, a therapeutic effect may include but is not limited to, one or more of (a) an immune-related response, as known to those skilled in the art as an immune-related complete response or an immune-related partial response relative to total tumor burden (e.g., an anti-tumor immune response); and (b) traditional overall objective response rate using the appropriate response assessment criteria known to those skilled in the art and depending on the type of cancer treated (e.g., for lymphoma, see Cheson et al., 2014, J. Clin. Oncology 32 (27): 3059-3067; for solid nonlymphoid tumors, Response Evaluation Criteria In Solid Tumors (RECIST)) (e.g., an antitumor response).

The term “cancer” is used herein to refer to all types of cancer, neoplasm or malignant tumors found in mammals (e.g., humans), including leukemias, lymphomas, carcinomas and sarcomas. Exemplary cancers that may be treated with a composition, combination or method provided herein include solid, non-lymphoid tumors, B cell leukemias, Non-Hodgkin's Lymphoma, and multiple myeloma.

The term “solid, non-lymphoid tumor” is used herein, for purposes of the specification and claims, to mean any primary tumor of epithelial cell origin, including tumors originating in an organ or gland such as liver, lung, brain, adrenal gland, breast, colon, bladder, pancreas, stomach, prostate, gastrointestinal tract, or reproductive tract (cervix, ovaries, endometrium etc.), or metastases thereof. For the purposes of the present invention, “solid, non-lymphoid tumor” also includes melanoma.

The phrase “therapeutically effective amount” means an amount of a composition or combination that results in a therapeutic effect following administration to an individual in need of such composition or combination. In immunotherapy, the therapeutic effect may be represented by activation of a T cell response that is suppressed prior to treatment with a method described herein. Such activation may be measured by an increase in one or more T cell subpopulations (e.g., CD4⁺ T cells, CD8⁺ T cells) using methods known in the art (e.g., labeling with detectable markers followed by flow cytometry analyses) or of the induced or increased expression of activation markers for such T cell subpopulations (e.g., increased CD44 or decreased CD62L expression). Alternatively, activation may also be measured by a decrease in the number or function of regulatory T cells (e.g., CD25⁺FoxP3⁺, CD4⁺ cells). Thus, in one aspect, therapeutic efficacy may be assessed by clinical outcome; an increase in the number of anti-tumor T cells or activated T cells as compared with the number prior to treatment or in absence of treatment with a combination of an antibody that binds human QSOX1 and an immune checkpoint inhibitor.

The term “individual” is used herein to refer to a mammal, preferably a human; and more preferably, a human in need of treatment with either an antibody that binds human QSOX1, or a combination of such antibody with an immune checkpoint inhibitor. The term individual may be used interchangeably with subject and/or patient.

Methods for treating cancer or initiating, enhancing, or prolonging an anti-tumor or anti-cancer response in an individual are provided herein. The methods may include administering an antibody provided herein to a subject to treat a cancer. In any of the methods of treatment provided herein, the dosage of an antibody will depend on such factors as the mode of administration, the formulation for administration, type of cancer, stage of cancer, the size and health of the individual to receive such a composition, and other factors which can be taken into consideration by a medical practitioner whom is skilled in the art of determining appropriate dosages for treatment. For example, for methods of treatment provided herein, an antibody may be administered in a dosage range (per body weight of the individual) that is between about 0.1 mg/kg to about 50 mg/kg, about 0.5 mg/kg to about 20 mg/kg, about 0.5 mg/kg to about 10 mg/kg, about 0.5 mg/kg to about 5 mg/kg, or 0.5 mg/kg to about 1 mg/kg. One skilled in the art can apply known principles and models of drug delivery and pharmacokinetics to ascertain a likely range of dosages to be tested in preclinical and clinical studies for determining a therapeutically effective amount of a composition or combination used in the methods of treatment provided herein. A composition or combination, useful in a method of treatment provided herein, may further comprise a pharmaceutically acceptable carrier to facilitate one or more of storage, stability, administration, and delivery. The carrier may be particulate, so that the composition or combination may be in, for example, powder or solid form. The carrier may be in a semi-solid, gel, or liquid formula, so that the composition or combination may be injected, applied, or otherwise administered. The mode of administration of a composition or combination, useful in a method of treatment provided herein, to an individual (such as a human) in need of thereof may be any mode known in the art to be suitable for delivering a pharmaceutical composition, and particularly suitable for treating cancer. A mode of administration may include but is not limited to, intravenously, intraperitoneally, subcutaneously, intramuscularly, by perfusion, and by peristaltic techniques. A composition or combination, useful in a method of treatment provided herein, may also be combined with other cancer treatments known to those skilled in the art, including but not limited to chemotherapeutic treatment and radiation therapies.

In a method of treatment provided herein, an antibody may be administered to an individual at a suitable frequency to be therapeutically effective. For example, an antibody may be administered once, more than once or over the course of multiple days or even weeks.

Certain embodiments of the invention are below, numbered 1-20.

1. An anti-QSOX1 antibody comprising a heavy chain variable region and a light chain variable region, wherein the heavy chain variable region (“VH”) comprises three complementarity determining regions, VH CDR1, VH CDR2, and VH CDR3 and the light chain variable region (“VL”) comprises three complementarity determining regions, VL CDR1, VL CDR2, and VL CDR3; and wherein the VH CDR1 comprises a sequence selected from the group consisting of SEQ ID NO: 1, SEQ ID NO: 7, and a sequence having at least 87% identity to SEQ ID NO: 1 or SEQ ID NO: 7; the VH CDR2 comprises a sequence selected from the group consisting of SEQ ID NO: 2, SEQ ID NO: 8, and a sequence having at least 85% identity to SEQ ID NO: 2 or SEQ ID NO: 8; the VH CDR3 comprises a sequence selected from the group consisting of SEQ ID NO: 3, SEQ ID NO: 9, and a sequence having at least 88% identity to SEQ ID NO: 3 or SEQ ID NO: 9; the VL CDR1 comprises a sequence selected from the group consisting of SEQ ID NO: 4, SEQ ID NO: 10, and a sequence having at least 83% identity to SEQ ID NO: 4 or SEQ ID NO: 10; the VL CDR2 comprises a sequence selected from the group consisting of SEQ ID NO: 5 and SEQ ID NO: 11; and the VL CDR3 comprises a sequence selected from the group consisting of SEQ ID NO: 6, SEQ ID NO: 12, and a sequence having at least 88% identity to SEQ ID NO: 6 or SEQ ID NO: 12.

2. The antibody of embodiment 1, wherein the antibody is capable of binding human QSOX1 with a K_{D} less than 10′ M.

3. The antibody of embodiment 1 or embodiment 2, wherein the antibody comprises: the VH CDR1 comprising SEQ ID NO: 1 or a sequence having at least 87% identity thereto, the VH CDR2 comprising SEQ ID NO: 2 or a sequence having at least 85% identity thereto, the VH CDR3 comprising SEQ ID NO: 3 or a sequence having at least 90% identity thereto, the VL CDR1 comprising SEQ ID NO: 4 or a sequence having at least 83% identity thereto, the VL CDR2 comprising SEQ ID NO: 5, and the VL CDR3 comprising SEQ ID NO: 6 or a sequence having at least 88% identity thereto.

4. The antibody of any one of embodiments 1-2, wherein the antibody comprises: the VH CDR1 comprising SEQ ID NO: 7 or a sequence having at least 87% identity thereto, the VH CDR2 comprising SEQ ID NO: 8 or a sequence having at least 90% identity thereto, the VH CDR3 comprising SEQ ID NO: 9 or a sequence having at least 88% identity thereto, the VL CDR1 comprising SEQ ID NO: 10 or a sequence having at least 90% identity thereto, the VL CDR2 comprising SEQ ID NO: 11, and the VL CDR3 comprising SEQ ID NO: 12 or a sequence having at least 88% identity thereto.

5. The antibody of any one of embodiments 1-2, wherein the antibody comprises the VH comprising a sequence selected from the group consisting of SEQ ID NO: 13, SEQ ID NO: 15, and a sequence having at least 90% identity to SEQ ID NO:13 or SEQ ID NO: 15; and the VL comprising a sequence selected from the group consisting of SEQ ID NO: 14, SEQ ID NO: 16, and a sequence having at least 90% identity to SEQ ID NO: 14 or SEQ ID NO: 16.

6. The antibody of embodiment 5, wherein the antibody comprises the VH comprising SEQ ID NO: 13 and the VL comprising SEQ ID NO: 14.

7. The antibody of embodiment 5, wherein the antibody comprises the VH comprising SEQ ID NO: 15 and the VL comprising SEQ ID NO: 16.

8. The antibody of any one of embodiments 1-7, wherein the heavy chain variable region (“VH”) comprises three complementarity determining regions, VH CDR1, VH CDR2, and VH CDR3, and four framework regions, VH FW1, VH FW2, VH FW3, and VH FW4, in the order VH FW1-VH CDR1-VH FW2-VH CDR2-VH FW3-VH CDR3-VH FW4; and wherein the light chain variable region (“VL”) comprises three complementarity determining regions, VL CDR1, VL CDR2, and VL CDR3, and four framework regions, VL FW1, VL FW2, VL FW3, and VL FW4, in the order VL FW1-VL CDR1-VL FW2-VL CDR2-VL FW3-VL CDR3-VL FW4.

9. The antibody of any one of embodiments 1-8, wherein the antibody comprises an Fc portion of a human or humanized antibody.

10. The antibody of any one of embodiments 1-9, wherein the antibody is attached to a solid support.

11. A composition comprising an antibody according to any one of embodiments 1-10 and an additional agent.

12. A method for treating cancer, the method comprising administering a therapeutically effective amount of an antibody according to any one of embodiments 1-9 to a subject in need thereof.

13. A method of determining the level of QSOX in a sample, the method comprising obtaining a sample from an individual and determining the level of QSOX1 in the sample using an antibody according to any one of embodiments 1-10.

14. The method of embodiment 13, wherein the sample is a blood sample.

15. The method of embodiment 14, wherein the sample is a plasma sample.

16. The method of any one of embodiments 13-15, wherein the determining step comprises an antigen capture assay comprising the antibody.

17. The method of embodiment 16, wherein the antibody comprises a detectable label.

18. The method of any one of embodiments 13-17, wherein a level of QSOX1 greater than ng/mL correlates with cancer in the subject.

19. A method of detecting QSOX1, the method comprising incubating a sample with an antibody according to any one of embodiments 1-9 attached to a solid support.

20. The method of embodiment 19, wherein the solid support is selected from a slide, tissue culture plate, tissue culture well, and a bead.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

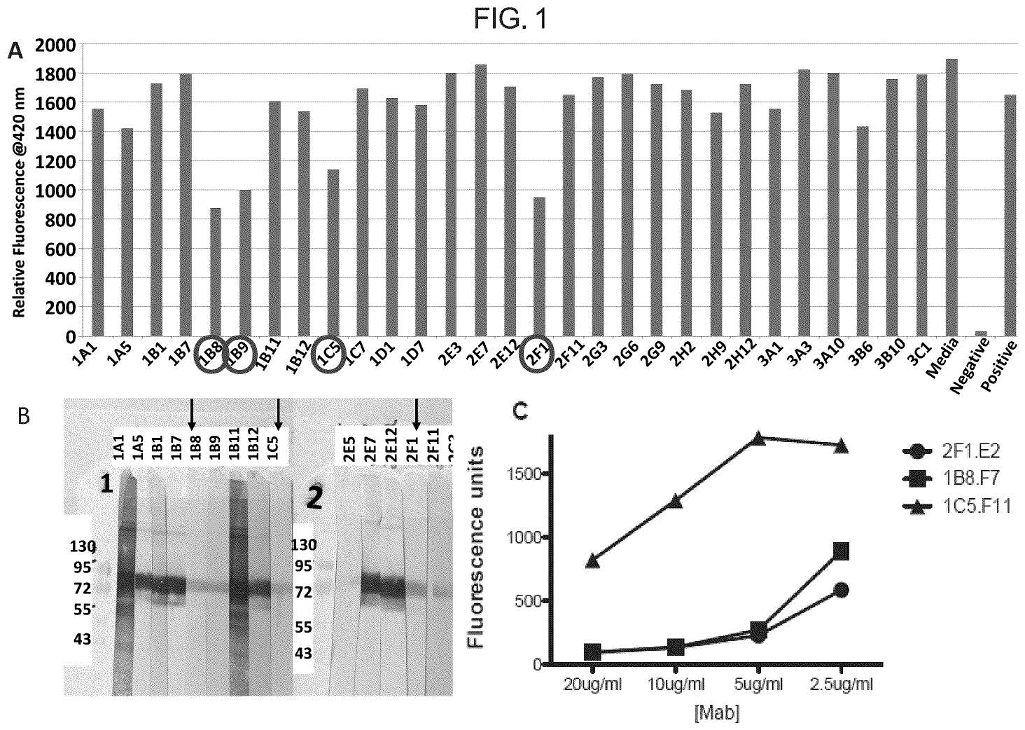

FIG. 1 shows the screening and identification of antibodies specific for QSOX1. The antibodies were screened for those that could block QSOX1 enzymatic activity in a fluorescent assay and those that could also bind directly to QSOX1 in a Western blot. The inhibition of QSOX1 activity was concentration dependent as shown in FIG. 1-C.

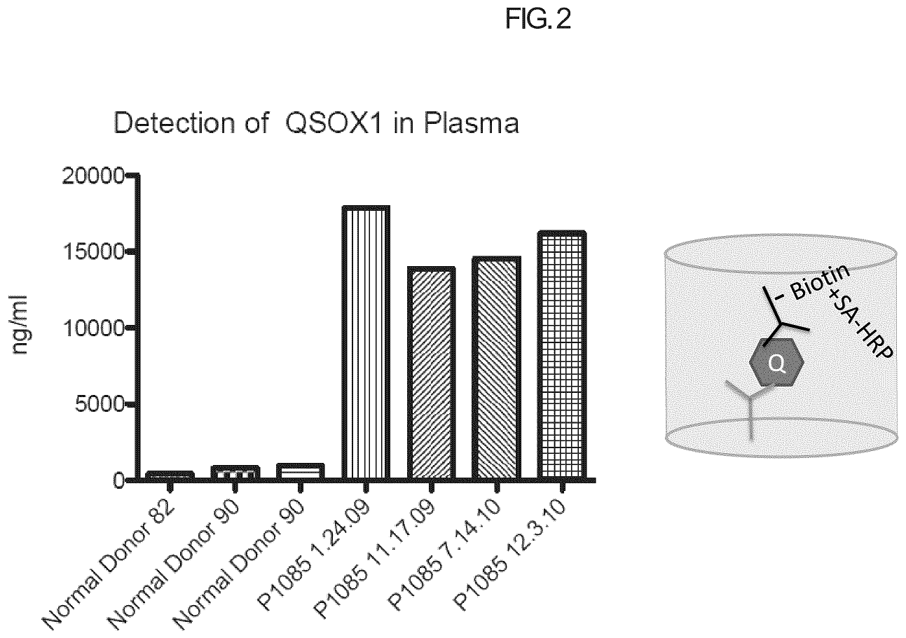

FIG. 2 shows a cartoon depiction of the QSOX1 antigen capture assay showing antibody 3A10 used to coat the plate followed by incubation with the sample and binding the QSOX1 in the sample to capture the antigen. The 2F1 antibody was labeled with biotin and was added to allow detection of the captured antigen. As can be seen in the graph, individuals with cancer had significantly higher levels of QSOX1 in their plasma as compared to normal donors. This assay could be the basis for a diagnostic or could be used to monitor subjects during treatment for the cancer or after treatment to monitor for recurrence.

To perform the assay, mAb 3A10 is coated onto an ELISA plate after which the plate is blocked with a non-specific protein (BSA). Dilutions of human plasma are then added to the 3A10-coated plate. The plasma is incubated in the plate for 30 minutes to one hour. The plate is then washed free of unbound plasma proteins. Biotinylated mAb 2F1 is added in an appropriate buffer to detect QSOX1 protein that has bound to mAb 3A10. The plate is further incubated for 30 minutes to one hour after which the plate is washed free of unbound biotinylated mAb 2F1. Streptavidin coupled to horse radish peroxidase (HRP) at an appropriate dilution in an appropriate buffer is then added to the plate and incubated and washed as before. Tetramethyl benzidine (TMB) substrate is added and the color in the plate is allowed to develop for 15 to 30 minutes after which 1 M H2SO4 is added to the plate to stop HRP. The optical densities of each well in the plate are read at O.S 450 nm. Levels of QSOX1 in patient plasma are calculated using a standard curve of recombinant QSOX1 from 50 ng/mL to less than 1 ng/mL.

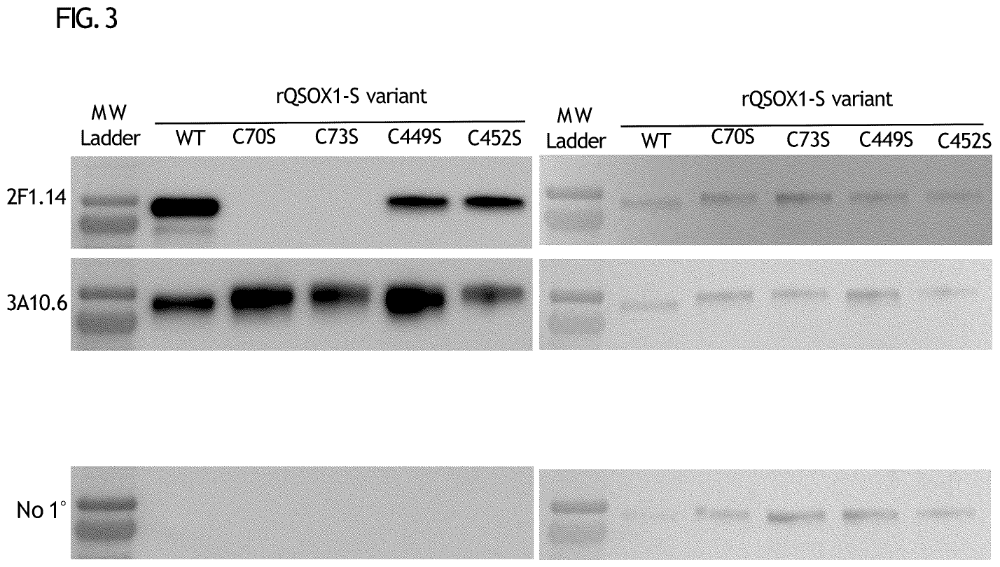

FIG. 3 shows a picture of a blot showing that the 2f1 antibody binds to the CxxC region of QSOX1 and the 3A10 antibody binds to a different region.

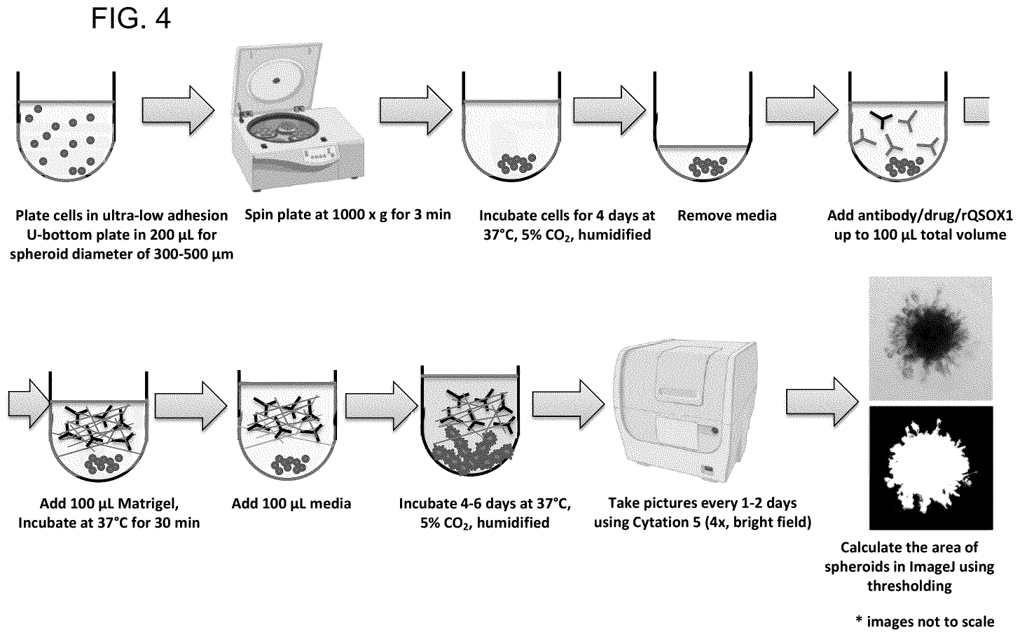

FIG. 4 shows the method of performing the 3D invasion assay, and FIG. 5 shows the results of that assay using the antibodies developed herein. In particular, 2F1 block 3D invasion in this assay with statistical significance.

TABLE 1
Sequences corresponding to
SEQ ID NOs referenced herein:

| SEQ | | IMGT |
|---|---|---|
| ID NO: | Sequence | CDR region |
| 1 | GFSLTDYG | 2F1; CDR1 |
| | | VH |
| 2 | IWGDGST | 2F1; CDR2 |
| | | VH |
| 3 | AGALYDGYYGY | 2F1; CDR3 |
| | | VH |
| 4 | QDVSTA | 2F1; CDR1 |
| | | VL |
| 5 | SAS | 2F1; CDR2 |
| | | VL |
| 6 | QQHYSTPLT | 2F1; CDR3 |
| | | VL |
| 7 | GLTFSDAW | 3A10; |
| | | CDR1 VH |
| 8 | IRSKAHNHAT | 3A10; |
| | | CDR2 VH |
| 9 | TRYGNPFVY | 3A10; |
| | | CDR3 VH |
| 10 | SPSLLYSSNQKNY | 3A10; |
| | | CDR1 VL |
| 11 | WAS | 3A10; |
| | | CDR2 VL |
| 12 | QQYYSYPWT | 3A10; |
| | | CDR3 VL |
| 13 | EVQLEESGPGLVAPSQSLSITCTVSGFSLTDYG | 2F1; VH |
| | VTWIRQPPGKGLEWLGVIWGDGSTYYNSALKSR | |
| | LSISKDDSKSQVFLKMNSLQTDDTAMYYCAGAL | |
| | YDGYYGYWGQGTTLTVSS | |
| 14 | DIVLTQTHKEMSTSVGDRVSITCKASQDVSTAV | 2F1; CDR3 |
| | AWYQQKPGQSPKLLIYSASYRYSGVPDRFTGSG | VL |
| | SGTDFTFTISSVQAEDLAVYYCQQHYSTPLTEG | |
| | AGTKLELK | |
| 15 | EVQLQQSGGGLVQPGGSMKLSCAASGLTFSDAW | 3A10 VH |
| | MDWVRQSPEKGLEWVAEIRSKAHNHATYYAESV | |
| | KGRFTISRDDSKSSVYLQMNSLRAEDTGIYYCT | |
| | RYGNPFVYWGQGTLVTVSA | |
| 16 | DIVMTQSPSSLAVSVGEKVIMSCKSSPSLLYSS | 3A10 VL |
| | NQKNYLAWYQQKPGQSPKLLIYWASTRESGVPD | |
| | RFTGSGSGTDFTLTISSVKAEDLAVYYCQQYYS | |
| | YPWTFGGGTKLEIK | |

What is claimed is:

1. An anti-QSOX1 antibody comprising a heavy chain variable region and a light chain variable region, wherein the heavy chain variable region (“VH”) comprises three complementarity determining regions, VH CDR1, VH CDR2, and VH CDR3 and the light chain variable region (“VL”) comprises three complementarity determining regions, VL CDR1, VL CDR2, and VL CDR3; and wherein
the VH CDR1 comprises a sequence selected from the group consisting of SEQ ID NO: 1, SEQ ID NO: 7, and a sequence having at least 87% identity to SEQ ID NO: 1 or SEQ ID NO: 7;
the VH CDR2 comprises a sequence selected from the group consisting of SEQ ID NO: 2, SEQ ID NO: 8, and a sequence having at least 85% identity to SEQ ID NO: 2 or SEQ ID NO: 8;
the VH CDR3 comprises a sequence selected from the group consisting of SEQ ID NO: 3, SEQ ID NO: 9, and a sequence having at least 88% identity to SEQ ID NO: 3 or SEQ ID NO: 9;
the VL CDR1 comprises a sequence selected from the group consisting of SEQ ID NO: 4, SEQ ID NO: 10, and a sequence having at least 83% identity to SEQ ID NO: 4 or SEQ ID NO: 10;
the VL CDR2 comprises a sequence selected from the group consisting of SEQ ID NO: 5 and SEQ ID NO: 11; and
the VL CDR3 comprises a sequence selected from the group consisting of SEQ ID NO: 6, SEQ ID NO: 12, and a sequence having at least 88% identity to SEQ ID NO: 6 or SEQ ID NO: 12.

2. The antibody of claim 1, wherein the antibody is capable of binding human QSOX1 with a K_{D} less than 10⁻⁶ M.

3. The antibody of claim 1, wherein the antibody comprises:
the VH CDR1 comprising SEQ ID NO: 1 or a sequence having at least 87% identity thereto,
the VH CDR2 comprising SEQ ID NO: 2 or a sequence having at least 85% identity thereto,
the VH CDR3 comprising SEQ ID NO: 3 or a sequence having at least 90% identity thereto,
the VL CDR1 comprising SEQ ID NO: 4 or a sequence having at least 83% identity thereto,
the VL CDR2 comprising SEQ ID NO: 5, and
the VL CDR3 comprising SEQ ID NO: 6 or a sequence having at least 88% identity thereto.

4. The antibody of claim 1, wherein the antibody comprises:
the VH CDR1 comprising SEQ ID NO: 7 or a sequence having at least 87% identity thereto,
the VH CDR2 comprising SEQ ID NO: 8 or a sequence having at least 90% identity thereto,
the VH CDR3 comprising SEQ ID NO: 9 or a sequence having at least 88% identity thereto,
the VL CDR1 comprising SEQ ID NO: 10 or a sequence having at least 90% identity thereto,
the VL CDR2 comprising SEQ ID NO: 11, and
the VL CDR3 comprising SEQ ID NO: 12 or a sequence having at least 88% identity thereto.

5. The antibody of claim 1, wherein the antibody comprises the VH comprising a sequence selected from the group consisting of SEQ ID NO: 13, SEQ ID NO: 15, and a sequence having at least 90% identity to SEQ ID NO:13 or SEQ ID NO: 15; and the VL comprising a sequence selected from the group consisting of SEQ ID NO: 14, SEQ ID NO: 16, and a sequence having at least 90% identity to SEQ ID NO: 14 or SEQ ID NO: 16.

6. The antibody of claim 5, wherein the antibody comprises the VH comprising SEQ ID NO: 13 and the VL comprising SEQ ID NO: 14.

7. The antibody of claim 5, wherein the antibody comprises the VH comprising SEQ ID NO: 15 and the VL comprising SEQ ID NO: 16.

8. The antibody of claim 1, wherein the heavy chain variable region (“VH”) comprises three complementarity determining regions, VH CDR1, VH CDR2, and VH CDR3, and four framework regions, VH FW1, VH FW2, VH FW3, and VH FW4, in the order VH FW1-VH CDR1-VH FW2-VH CDR2-VH FW3-VH CDR3-VH FW4; and wherein the light chain variable region (“VL”) comprises three complementarity determining regions, VL CDR1, VL CDR2, and VL CDR3, and four framework regions, VL FW1, VL FW2, VL FW3, and VL FW4, in the order VL FW1-VL CDR1-VL FW2-VL CDR2-VL FW3-VL CDR3-VL FW4.

9. The antibody of claim 8, wherein the antibody comprises an Fc portion of a human or humanized antibody.

10. The antibody of claim 1, wherein the antibody is attached to a solid support.

11. A composition comprising an antibody according to claim 1 and an additional agent.

12. A method for treating cancer, the method comprising administering a therapeutically effective amount of an antibody according to claim 1 to a subject in need thereof.

13. A method of determining the level of QSOX in a sample, the method comprising obtaining a sample from an individual and determining the level of QSOX1 in the sample using an antibody according to claim 1.

14. The method of claim 13, wherein the sample is a blood sample.

15. The method of claim 14, wherein the sample is a plasma sample.

16. The method of claim 13, wherein the determining step comprises an antigen capture assay comprising the antibody.

17. The method of claim 16, wherein the antibody comprises a detectable label.

18. The method of claim 13, wherein a level of QSOX1 greater than 5,000 ng/mL correlates with cancer in the subject.

19. A method of detecting QSOX1, the method comprising incubating a sample with an antibody according to claim 1 attached to a solid support.

20. The method of claim 19, wherein the solid support is selected from a slide, tissue culture plate, tissue culture well, and a bead.
